(19) United States
(12) Patent Application Publication
(10) Pub. No.: US 2026/0364331 A1
(43) Pub. Date: Nov. 19, 2026

(54) Tamper Resistant Closed-System Drug Transfer Device Connector

(71) Applicant: Becton, Dickinson and Company, Franklin Lakes, NJ (US)

(72) Inventor: Jason Andrew Wine, Brea, CA (US)

(21) Appl. No.: 19/212,079

(22) Filed: May 19, 2025

(51) Int. Cl. A61M 39/10

(52) U.S. Cl. CPC A61M 39/10; A61M 39/1033; A61M 2039/1083; A61M 2039/1088; A61M 2207/00

(57) ABSTRACT

Provided herein is a connector comprising a central shaft defining a connector lumen, a seal positioned in a proximal opening of the central shaft, and a luer housing at a distal end of the central shaft defining a cavity. A male luer connector is positioned within the cavity and is rotatable relative to the housing. The male luer connector and the housing comprise a ratchet and pawl system that enables rotation between the male luer connector and housing when the housing is turned in a first direction and prevents rotation between the male luer connector and housing when the housing is turned in a second direction. A pair of finger tabs on the housing may be deflected radially inward to engage the male luer connector and prevent rotation between the male luer connector and housing when the housing is turned in the first direction.

### BACKGROUND OF THE INVENTION

### Field of the Invention

The present disclosure relates generally to closed-system drug transfer devices and, more specifically, to features of closed-system drug transfer devices that provide for controlled connection and disconnection of components thereof.

### Description of Related Art

Healthcare providers and medical device and pharmaceutical manufacturers, as well as, leading governmental and regulatory agencies, have long recognized the issues healthcare workers face with compounding and administering hazardous drugs. For example, nurses treating cancer patients risk being exposed to chemotherapy drugs when compounding and administering these drugs, and this unintentional chemotherapy exposure can affect the nervous system, impair the reproductive system, and/or bring an increased risk of developing blood cancers in the future.

In order to reduce the risk of healthcare workers being exposed to hazardous drugs, the closed transfer of these drugs becomes important, with various policies and regulations having been implemented that require the use of closed-system drug transfer devices (CSTDs) when compounding and administering hazardous drugs. A CSTD may provide for the transfer of hazardous drugs out from, between, or through syringes, vials, intravenous (IV) fluid bags, and IV lines, as non-limiting examples, with the CSTD providing for a safe removal, transfer, and/or administration of the hazardous drug. The CSTD may utilize a physical barrier that inhibits the escape of the hazardous drug substance to thereby mitigate the exposure risk to the healthcare worker who may compound or administer hazardous drugs to patients.

A typical CSTD as known in the art may include a fluid container injector, such as a BD PhaSeal™ Optima Injector from Becton, Dickinson and Company, as a non-limiting example. The injector may comprise a female luer connection at one end that may be coupled to the male luer tip of a syringe or a male luer connection of an IV line, as non-limiting examples. The injector may further comprise an inner cannula that engages and pierces a seal of a connector inserted into the injector, to provide a fluid path through the injector and connector, with it recognized that the connector may be provided as a BD PhaSeal™ Optima Infusion Connector C35-O, as a non-limiting example. Use of an injector and connector as part of a CSTD provides substantially leak-proof sealing during engagement of multiple components, during transfer of a hazardous substance between such components, and during disengagement of the components.

In practice, a CSTD that includes an injector and connector may transfer fluid from/between a fluid source coupled to the injector and a fluid conduit (e.g., IV line) coupled to the connector. The coupling between the connector and the IV line may be made via connection of a male luer connection of the connector and a needle free connector of the IV line. For longer term infusions (e.g., approximately over a day), the CSTD and associated fluid source/IV line may be sent home with the patient. It is critical in this scenario that the connector remain attached and not become loose from the IV line. Since the connector contains a simple male luer lock interface, it is possible that it may become loose from the IV line, either intentionally or unintentionally, resulting in unwanted exposure and delay of therapy. However, when a patient's infusion is complete, it is often required that the connector be removed from the IV line, in order to allow access to the clinician or patient to flush the IV line of drug.

Accordingly, a need exists for the connector of a CSTD to have a tamperproof connection that can aid in preventing unwanted disconnection, but that can also be overridden when intended.

### SUMMARY OF THE INVENTION

Provided herein is a closed system drug transfer device connector for transferring a drug. The connector comprises a central shaft defining a connector lumen, with the central shaft including flanges extending out radially therefrom and a pair of flexible fingers each comprising a proximal end attached to the central shaft and a free distal end configured to deflect radially inward and outward. The connector also comprises a seal positioned in a proximal opening of the central shaft and a luer housing formed integrally with the central shaft and positioned at a distal end of the central shaft, the luer housing comprising a sidewall defining a cavity therein, with the sidewall including a pair of flexible finger tabs formed thereon configured to deflect radially inward responsive to a pinching force applied thereto. The connector further comprises a male luer connector positioned within the cavity so as to be selectively rotatable relative to the luer housing. The male luer connector and the luer housing comprise a ratchet and pawl system configured to allow for rotation of the male luer connector relative to the luer housing when the luer housing is turned in a first direction but prevent rotation of the male luer connector relative to the luer housing when the luer housing is turned in a second direction. With the pair of finger tabs deflected radially inward, the pair of finger tabs engage the male luer connector to prevent rotation of the male luer connector relative to the luer housing when the luer housing is turned in the first direction.

In some embodiments, the male luer connector comprises a distal male luer connection including an elongated protrusion and a collar surrounding the elongated protrusion, a stem extending proximally away from the distal male luer connection, and a luer lumen extending through the elongated protrusion and through the stem.

In some embodiments, the luer housing comprises an inner surface including a first annular protrusion that defines a receptacle within the cavity, the receptacle configured to receive a proximal end of the stem therein, with the luer lumen aligned and in fluid communication with the connector lumen when the stem is positioned in the receptacle.

In some embodiments, the stem includes a groove formed in an outer surface thereof, with an O-ring positioned in the groove, the O-ring forming a seal with the first annular protrusion, to provide a leak-proof connection between the connector lumen and the luer lumen.

In some embodiments, the luer housing comprises an inner surface including a second annular protrusion that defines an inner lip, and wherein the male luer connector comprises an annular lip formed on the collar, with the inner lip and the annular lip providing a snap-fit connection that retains the male luer connector within the cavity, while allowing for rotation of the male luer connector relative to the luer housing.

In some embodiments, the collar includes a plurality of ribs formed on an outer surface thereof, the plurality of ribs spaced circumferentially around the collar and extending radially out therefrom, and wherein the pair of finger engage with a portion of the plurality of ribs when deflected radially inward.

In some embodiments, the ratchet and pawl system comprises a plurality of pawl teeth formed on an inner surface of the luer housing at a location proximal to the pair of finger tabs and a plurality of ratchet teeth formed on an outer surface of the stem, wherein the plurality of pawl teeth and the plurality of ratchet teeth each comprise angled teeth that interact to allow for rotation of the male luer connector relative to the luer housing when the luer housing is turned in the first direction but prevent rotation of the male luer connector relative to the luer housing when the luer housing is turned in the second direction.

In some embodiments, the ratchet and pawl system comprises a plurality of ratchet teeth formed on an inner surface of the luer housing at a location proximal to the pair of finger tabs and a plurality of pawl teeth formed on an outer surface of the stem, wherein the plurality of pawl teeth and the plurality of ratchet teeth each comprise angled teeth that interact to allow for rotation of the male luer connector relative to the luer housing when the luer housing is turned in the first direction but prevent rotation of the male luer connector relative to the luer housing when the luer housing is turned in the second direction.

In some embodiments, the plurality of pawl teeth and the plurality of ratchet teeth each comprise a pair of pawl teeth and a pair of ratchet teeth, respectively.

In some embodiments, each of the pair of finger tabs comprises a cantilevered tab having a first end attached to the sidewall and a free second end.

In some embodiments, the first direction comprises a counterclockwise direction and the second direction comprises a clockwise direction.

Also provided herein is a closed system drug transfer device including a connector and a fluid source, container, or conduit. The connector comprises a central shaft defining a connector lumen, with the central shaft including flanges extending out radially therefrom and a pair of flexible fingers each comprising a proximal end attached to the central shaft and a free distal end configured to deflect radially inward and outward. The connector also comprises a seal positioned in a proximal opening of the central shaft and a luer housing formed integrally with the central shaft and positioned at a distal end of the central shaft, the luer housing comprising a sidewall defining a cavity therein, with the sidewall including a pair of flexible finger tabs formed thereon configured to deflect radially inward responsive to a pinching force applied thereto. The connector further comprises a male luer connector positioned within the cavity so as to be selectively rotatable relative to the luer housing. The male luer connector and the luer housing comprise a ratchet and pawl system configured to allow for rotation of the male luer connector relative to the luer housing when the luer housing is turned in a first direction but prevent rotation of the male luer connector relative to the luer housing when the luer housing is turned in a second direction. With the pair of finger tabs deflected radially inward, the pair of finger tabs engage the male luer connector to prevent rotation of the male luer connector relative to the luer housing when the luer housing is turned in the first direction. The fluid source, container, or conduit includes a female luer connection thereon, with the female luer connection configured to engage the male luer connector to mechanically and fluidly connect the connector to the fluid source, container, or conduit.

In some embodiments, the prevention of rotation of the male luer connector relative to the luer housing when the luer housing is turned in the second direction enables a twisting engagement between the male luer connector and the female luer connection when the luer housing is turned in the second direction.

In some embodiments, with the female luer connection engaged with the male luer connector, the allowing of rotation of the male luer connector relative to the luer housing when the luer housing is turned in the first direction prevents disconnection of the female luer connection from the male luer connector.

In some embodiments, with the female luer connection engaged with the male luer connector and with the pair of finger tabs deflected radially inward, the pair of finger tabs engage the male luer connector to prevent rotation of the male luer connector relative to the luer housing when the luer housing is turned in the first direction, such that turning of the luer housing in the first direction enables disconnection of the female luer connection from the male luer connector.

In some embodiments, the fluid source, container, or conduit comprises an IV line and a needle-free connector attached thereto, with the female luer connection provided on the needle-free connector.

Also provided herein is method for assembling and disassembling a closed system drug transfer device. The method includes pushing the male luer connector of a connector into engagement with the female luer connection of a fluid source, container, or conduit, and turning the luer housing in the second direction to thread the male luer connector into twisting engagement with the female luer connection.

In some embodiments, with the female luer connection engaged with the male luer connector, the method further comprises turning the luer housing in the first direction, with the male luer connector rotating freely relative to the luer housing when the luer housing is turned in the first direction, such that disconnection of the female luer connection from the male luer connector is prevented.

In some embodiments, with the female luer connection engaged with the male luer connector, the method further comprises applying a pinching force to the pair of finger tabs to cause the pair of finger tabs to deflect radially inward and, with the pair of finger tabs deflected radially inward, turning the luer housing in the first direction, with the pair of finger tabs engaging the male luer connector to prevent rotation of the male luer connector relative to the luer housing, such that turning of the luer housing in the first direction disconnects the female luer connection from the male luer connector.

### BRIEF DESCRIPTION OF THE DRAWINGS

FIG. 1 is a perspective view of a closed-system drug transfer device (CSTD) for transferring fluids between fluid containers or conduits, with the CSTD including an injector and connector as known in the art;

FIG. 2 is an exploded view of the injector included in the CSTD of FIG. 1;

FIG. 3 is a side view of a connector useable in a CSTD, according to an embodiment of the disclosure;

FIG. 4 is a side cross-sectional view of the connector of FIG. 3, taken along line 4-4;

FIG. 5 is a bottom view of the connector of FIG. 3;

FIG. 6 is a cross-sectional view of the connector of FIG. 3, taken along line 6-6, according to one embodiment of the disclosure;

FIG. 7 illustrates the connector of FIG. 3 being initially engaged with a needle free connector of an IV line, with the connector being rotated in a clockwise direction to facilitate engagement therebetween;

FIG. 8 illustrates rotation of the connector in a counterclockwise direction after connection to the IV line, with a housing of the connector slipping over a male luer connector of the connector, such that the male luer connector of the connector remains engaged with the female luer connection of the IV line;

FIG. 9 illustrates finger tabs of the connector being depressed radially inward, to cause the finger grips to engage the male luer connector of the connector;

FIG. 10 illustrates rotation of the connector in a counter-counterclockwise direction while the finger tabs are depressed, such that the male luer connector of the connector disengages from the female luer connection of the IV line, to enable removal of the connector from the IV line; and

FIG. 11 is a cross-sectional view of the connector of FIG. 3, taken along line 6-6, according to another embodiment of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with another component or with a patient. Thus, for example, the end of a device first contacting another component or the body 44 of the patient would be the distal end, while the opposite end of the device being manipulated by the user would be the proximal end of the device.

The terms “first”, “second”, and the like are not intended to refer to any particular order or chronology, but refer to different conditions, properties, or elements.

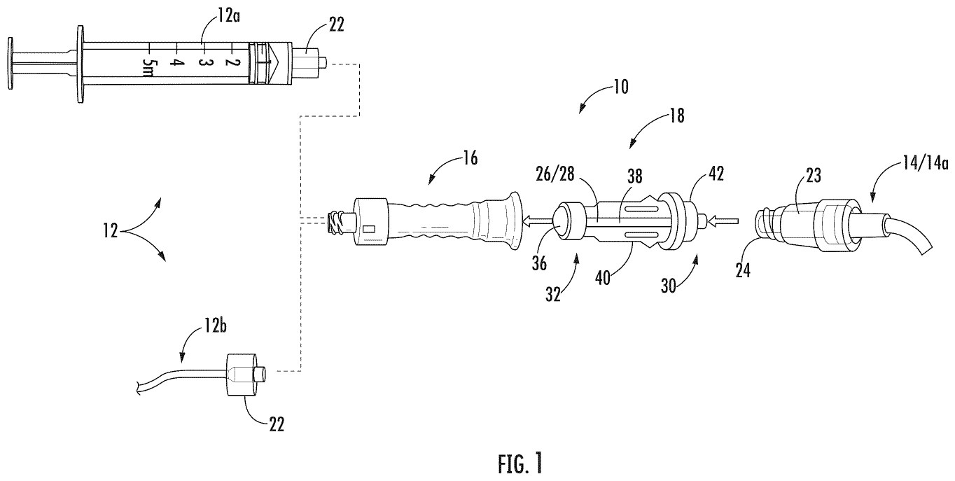

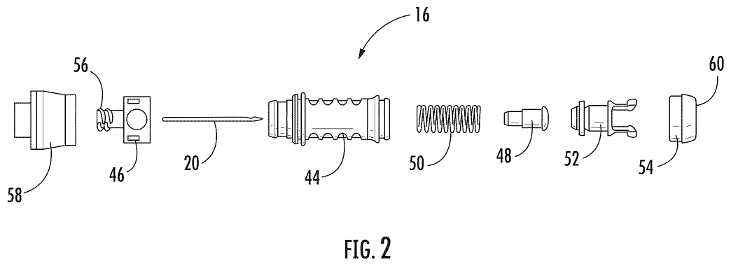

Referring to FIGS. 1 and 2, shown is the general arrangement of a system 10 for the closed transfer of fluids (i.e., a closed-system drug transfer device (CSTD) 10) with which aspects of the disclosure may implemented. In the illustrated embodiment, the CSTD 10 may provide for the closed transfer of fluids from/between a first fluid source, container, or conduit 12 and a second fluid source, container, or conduit 14. The CSTD 10 includes an injector 16 and a connector 18. The CSTD 10 provides substantially leak-proof sealing during engagement of a cannula 20 of the injector 16 with connector 18, during transfer of a substance from/between the first fluid source, container, or conduit 12 and the second fluid source, container, or conduit 14 that flows through the injector 16 and connector 18. The leak-proof sealing of the CSTD 10 substantially prevents leakage of both air and liquid during use of the CSTD 10.

In the non-limiting embodiment of FIG. 1, the first fluid source, container, or conduit 12 may be provided as a syringe 12a or intravenous (IV) line 12b that is connected to a proximal end of the injector 16. The syringe 12a or IV line 12b comprises a male luer connection 22 thereon that enables a mechanical and fluid connection with the injector 16. Also, in the non-limiting embodiment of FIG. 1, the second fluid source, container, or conduit 14 may be provided as an IV line 14a that is connected to a distal end of connector 18. The IV line 14a may include a needle-free connector (NFC) 23 attached thereto having a female luer connection 24, with the needle-free connector 23 providing a mechanical and fluid connection between the connector 18 and the IV line 14a.

As shown in FIG. 1, the connector 18 is configured to be secured to each of the injector 16 and the needle-free connector 23, to form an open fluid path therebetween. The connector 18 includes a central shaft 26 defining a lumen 28 that extends between a distal end 30 and a proximal end 32 of the connector 18. The lumen 28 ends at a proximal opening 34 provided at the proximal end 32 of the central shaft 26, and a self-closing or self-healing seal 36 is provided over the proximal opening 34 that may be pierced by a cannula 20 of the injector 16 (FIG. 2) to provide access to the lumen 28 when the injector 16 is connected to connector 18. The central shaft 26 may include an arrangement of flanges 38 and flexible fingers 40 that protrude radially outward therefrom and that mate with the injector 16 when the injector 16 is slid linearly into the connector 18. That is, while not shown in FIG. 1, it is recognized that the flanges 38 and flexible fingers 40 would engage with guiding grooves formed on an interior surface of the injector 16, with the snap fit fingers 40 flexing and forming a snap-fit engagement with a lip of the injector 16 to secure the connector 18 in the injector 16.

According to the embodiment of FIG. 1, a male luer connector 42 is provided at the distal end 30 of the central shaft 26. The male luer connector 42 comprises an elongated protrusion and a surrounding collar and is configured to engage the female luer connection 24 of the male luer connection 42, to mechanically and fluid connect the connector 18 to the IV line 14a.

As best shown in FIG. 2, an exemplary injector 16 includes a body 44, a luer hub 46, a septum 48, a spring 50, a connector clip 52, a (distal) end cap 54, and the cannula 20. The luer hub 46 is secured to the body 44 at a proximal end of the injector 16 and includes a female luer connection 56 configured to engage the male luer connection 22 of the syringe 12a or IV line 12b. The luer hub 46 may comprise a threaded outer surface, as well as a tapered luer cavity, with the cavity retaining the septum 48 therein. The luer hub 46 may also include a cannula hub (not shown) therein within which cannula 20 may be secured, with the cannula 20 extending out distally from the cannula hub and into a hollow interior of body 44. In some embodiments, the injector 16 may also include a hub cover 58 that may be secured to the proximal end of body 44 and that functions to secure the luer hub 46 on the body 44. The spring 50 is secured within the hollow interior of body 44, at the proximal end of the body 44. The connector clip 52 is secured to a distal end of the spring 50 and is positioned within the hollow interior of body 44. The connector clip 52 is configured to engage the proximal end of connector 18 when the connector 18 is pressed into engagement with injector 16, with the connector 18 engaging a shoulder of connector clip 52 when the connector 18 is fully engaged with injector 16. With the connector 18 fully engaged with injector 16, the cannula 20 penetrates through the seal 36 of connector 18, such that the cannula 20 is brought into fluid communication with the lumen 28 of connector 18—thereby forming a continuous fluid path extending from the luer hub 46 of injector 16, through connector 18, and to the female luer connection 24 of the second fluid source, container, or conduit 14, such that the first fluid source, container, or conduit 12 is fluidly connected to the second fluid source, container, or conduit 14 and a transfer of fluid therebetween is provided. The end cap 54 is formed integrally with or secured to the distal end of body 44 and functions to maintain engagement between the connector 18 and the injector 16 when the connector 18 is pressed into engagement with injector 16—with a lip 60 on end cap 54 engaging the flexible fingers 40 to secure the connector 18 to injector 16.

It is recognized that there is the potential for the CSTD 10—and specifically the connector 18—to disconnect from the second fluid source, container, or conduit 14 (e.g., IV line 14a) during use of the CSTD 10. That is, it is possible for the male luer connector 42 of connector 18 to accidentally disconnect from the female luer connection 24 of needle-free connector 23, such as via the male luer connector 42 of the connector 18 unscrewing from the female luer connection 24. Such a disconnection of the connector 18 from the IV line 14a can result in a patient or healthcare worker being exposed to hazardous drugs.

According to aspects of the disclosure, a connector of a CSTD 10 may be provided that is configured to prevent accidental disconnection between the connector and a second fluid source, container, or conduit (e.g., IV line) connected thereto. In particular, the connector is configured to enable achieving of a threaded connection between a male luer connector of the connector and a female luer connector of an external fluid adapter or conduit, with such connection provided responsive to tightening in the clock-wise (CW) direction, but with disconnection prevented when twisting occurs in the counter-clock-wise (CCW) direction. Additionally, the connector is configured to enable disconnection of the male luer connector of the connector from the female luer connection of an external adapter or conduit when a separate feature of the connector is engaged, and twisting is applied in the CCW direction.

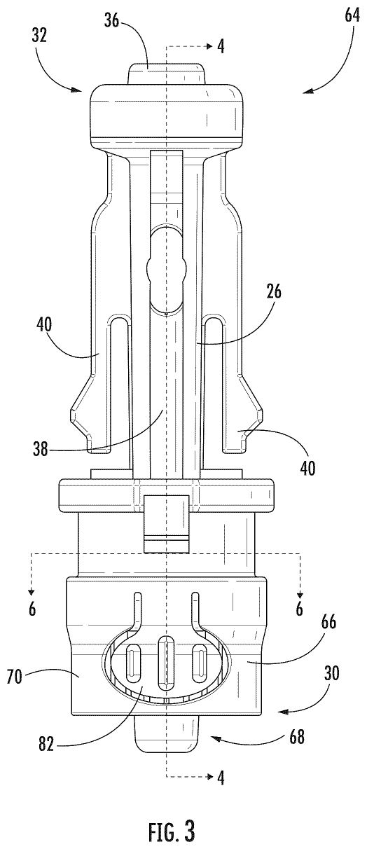

Referring to FIGS. 3-6, a connector 64 is provided that is configured to prevent accidental disconnection between the connector 64 and a second fluid source, container, or conduit 14, such as an IV line 14a as shown in FIG. 1. The connector 64 includes a number of features or components similar to the connector 18 shown and described in FIGS. 1 and 2, and thus like features/components are numbered identically, but the connector 64 includes features thereon that provide a controlled connection and disconnection from the IV line 14a.

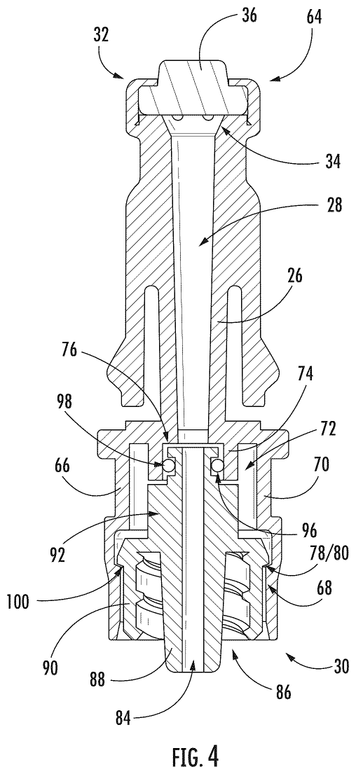

As best shown in FIGS. 3 and 4, the connector 64 includes a central shaft 26 defining a (connector) lumen 28 that extends lengthwise through at least a portion of the connector 64. The lumen 28 ends at a proximal opening 34 provided at the proximal end 32 of the central shaft 26, and a seal 36 is provided over the proximal opening 34 that may be pierced by the cannula 20 of the injector 16 (FIGS. 1 and 2) to provide access to the lumen 28 when the injector 16 is connected to connector 64. The central shaft 26 may include an arrangement of flanges 38 and flexible fingers 40 that protrude radially outward therefrom and that mate with the injector 16 when the injector 16 is slid linearly downward onto the connector 64—with the flanges 38 arranged perpendicular to the flexible fingers 40.

According to embodiments of the disclosure, the connector 64 further includes a luer housing 66 positioned at the distal end 30 of the central shaft 26 that is configured to receive and retain a male luer connector 68 therein. In some embodiments, the luer housing 66 may be integrally formed with the central shaft 26, while in other embodiments the luer housing 66 may be securely connected to the central shaft 26. The luer housing 66 has a generally cylindrical outer wall 70 that defines a cavity 72 therein that receives the male luer connector 68, with the cavity 72 in fluid communication with lumen 28. The luer housing 66 includes an arrangement of protrusions formed on an inner surface thereof, including a first annular protrusion 74 that defines a receptacle 76 within the cavity 72 and a second annular protrusion 78 that defines an inner lip 80.

Also included on the cylindrical outer wall 70 of luer housing 66 (and forming part of the cylindrical outer wall 70) is a pair of finger tabs 82 that are engageable by a user. The finger tabs 82 are formed on opposing sides of the cylindrical outer wall 70, with each of the finger tabs 82 comprising a cantilevered member having a fixed first end and a free second end. The cantilevered finger tabs 82 are configured as flexible tabs that may be deflected (radially) inward responsive to a pinching force being applied thereto by a user, with the finger tabs 82 selectively engaging the male luer connector 68 when deflected inward, as explained in further detail below.

As best shown in FIG. 6, a ratchet and pawl system 83 is collectively provided on the male luer connector 68 and luer housing 66 to provide for a selective rotation between the male luer connector 68 and luer housing 66. With regard to the luer housing 66, the luer housing 66 may further include a plurality of pawl teeth 84 formed thereon. The pawl teeth 84 are formed on an inner surface of the cylindrical outer wall 70, at a location proximal to the finger tabs 82. In the illustrated embodiment, two pawl teeth 84 are formed on an inner surface of the cylindrical outer wall 70, but it is recognized that a greater number of pawl teeth 84 could be provided-such as four or six pawl teeth 84, as further non-limiting examples (with two additional pawl teeth 84 shown in phantom in FIG. 6). Each of the pawl teeth 84 comprises an angled tooth that interacts with ratchet teeth on the male luer connector 68 to allow for rotation of male luer connector 68 relative to the luer housing 66 in one direction but prevent rotation of the male luer connector 68 relative to the luer housing 66 in the other direction, as explained in further detail below.

The male luer connector 68 includes a distal end that includes a male luer connection 86 comprising an elongated protrusion 88 and a surrounding collar 90 having a threaded inner surface, with the elongated protrusion 88 and collar 90 configured to engage a female luer connection, such as the female luer connection 24 on a needle free connector 23 attached to an IV line 14a (FIG. 1). In some embodiments, the opposing proximal end of the male luer connector 68 may comprise a stem 92 that extends proximally away from the collar 90 and protrusion 88, for engagement with the receptacle 76 formed within cavity 72 of luer housing 66, with the stem 92 being received in the receptacle 76 of luer housing 66 to align a (luer) lumen 94 of male luer connector 68 (that extends through elongated protrusion 88 and stem 92) with the lumen 28 of connector 64. The stem 92 is configured to include a groove 96 formed on an outer surface thereof into which an O-ring 98 may be placed, with the O-ring 98 forming a seal against the luer housing 66 in receptacle 76 to provide a leak-proof connection between the lumens 28, 94 in connector 64.

The male luer connector 68 also includes an annular lip 100 formed on an outer surface of the collar 90 thereof, with the annular lip 100 extending radially outward. The annular lip 100 on collar 90 is configured to interact with and engage the inner lip 80 formed on the inner surface of housing portion 66, to retain the male luer connector 68 within the cavity 72 of the housing portion 66. In particular, to secure the male luer connector 68 to/in luer housing 66, the male luer connector 68 may be pushed proximally into the cavity 72 of housing until the annular lip 100 on collar 90 is pushed proximally past the inner lip 80 of housing portion 66—with the male luer connector 68 thus being engaged with the luer housing 66 and secured thereto via a “snap-fit” connection. While the engagement of annular lip 100 and inner lip 80 prevents removal of the male luer connector 68 from the luer housing 66, the male luer connector 68 is allowed to selectively rotate within cavity 72 and relative to luer housing 66, as explained in further detail below.

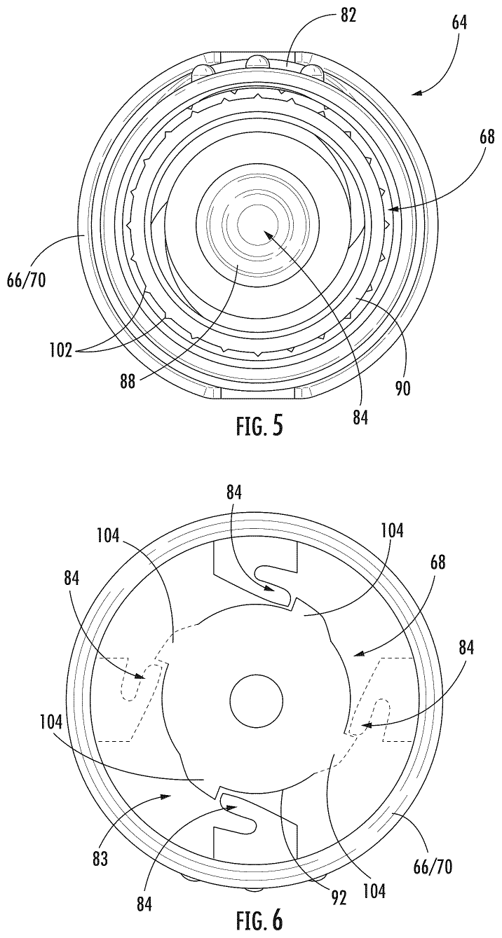

As best shown in FIG. 5, in some embodiments, the outer surface of the collar 90 of male luer connector 68 may comprise a plurality of ribs 102 formed thereon that extend out radially from the collar 90. The plurality of ribs 102 may be spaced apart circumferentially about the collar 90, with each of the ribs 102 extending along a length of the collar 90. As explained further below, the ribs 102 may interact with the finger tabs 82 of luer housing 66 when the finger tabs 82 are depressed inward, to provide for gripping of the male luer connector 68 with finger tabs 82.

As best shown in FIG. 6, the male luer connector 68 further includes a plurality of ratchet teeth 104 formed on the stem 92—i.e., on stem 92 at a location distal to where stem 92 in received within receptacle 76. In the illustrated embodiment, two ratchet teeth 104 are formed on an outer surface of the stem 92, but it is recognized that a greater number of ratchet teeth 104 could be provided-such as four or six ratchet teeth 104, as further non-limiting examples (with two additional ratchet teeth 104 shown in phantom in FIG. 6). Each of the ratchet teeth 104 comprises an angled tooth that is configured to interact with the pawl teeth 84 formed on the inner surface of the cylindrical outer wall 70 of housing portion 66 to selectively provide for rotation between the male luer connector 68 and the luer housing 66 of connector 64. Specifically, the pawl teeth 84 on luer housing 66 and the ratchet teeth 104 on the male luer connector 68 each comprise angled teeth that interact to allow for rotation of the male luer connector 68 relative to the luer housing 66 in one direction but prevent rotation of the male luer connector 68 relative to the luer housing 66 in the other direction.

With engagement of the male luer connector 68 and the luer housing 66 as described above—including interaction between the pawl teeth 84 on luer housing 66 and the ratchet teeth 104 on the male luer connector 68—the connector 64 is configured to allow for connection of the male luer connector 68 with a female luer connection 24 (of a needle-free connector 23 attached to an IV line 14a, as in FIG. 1) but prevent a subsequent disconnection of the male luer connector 68 from the female luer connection 24. That is, a rotation of the male luer connector 68 relative to the luer housing 66 may be prevented when grasping and turning the connector 64 in the clockwise direction based on a locking engagement between the pawl teeth 84 on luer housing 66 and the ratchet teeth 104 on the male luer connector 68, which allows for the male luer connector 68 (and overall connector 64) to be connected to a female luer connection 24 via application of a twisting force/motion to connector 64. Conversely, a rotation of the male luer connector 68 relative to the luer housing 66 may be allowed when grasping and turning the connector 64 in the counterclockwise direction based on a slipping of the pawl teeth 84 on luer housing 66 over the ratchet teeth 104 on the male luer connector 68, which prevents disconnection of the male luer connector 68 from the female luer connection 24, as any rotation of connector 64 is not transferred to male luer connector 68—resulting in the male luer connector 68 remaining engaged/secured to female luer connection 24.

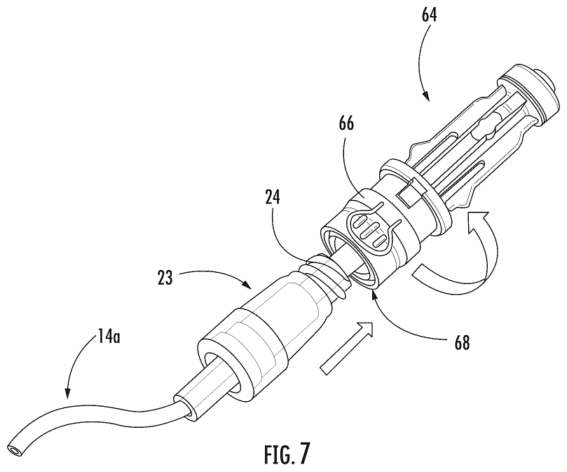

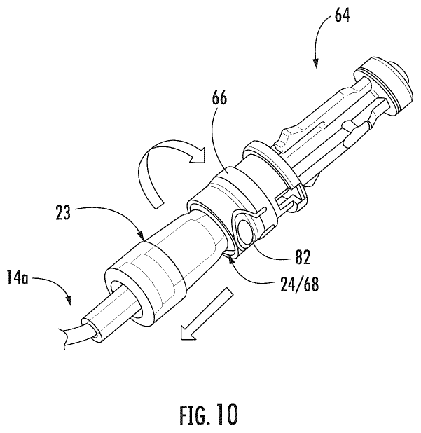

Referring now to FIGS. 7-10, connection and disconnection of the connector 64 to a needle-free connector 23 on an IV line 14a is illustrated, according to an aspect of the disclosure.

As shown first in FIG. 7, the connector 64 and IV line 14a are joined by pushing and twisting the male luer connector 68 of connector 64 and the female luer connector 24 of needle-free connector 23 into engagement with each other. In connecting the male luer connector 68 with the female luer connection 24, a twisting force/motion may be applied to the luer housing 66 of connector 64 in the clockwise direction, with a locking engagement between the pawl teeth 84 on luer housing 66 and the ratchet teeth 104 on the male luer connector 68 (FIG. 6) transmitting the twisting motion to the male luer connector 68 to enable connection thereof to the female luer connection 24.

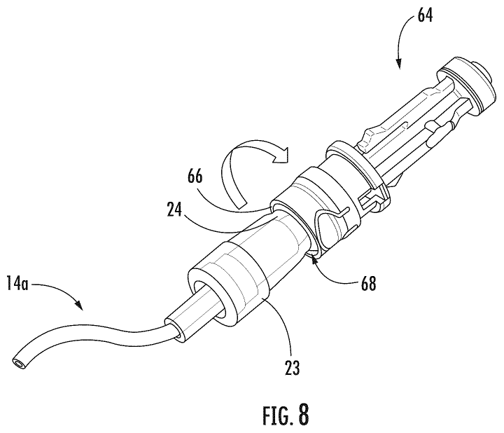

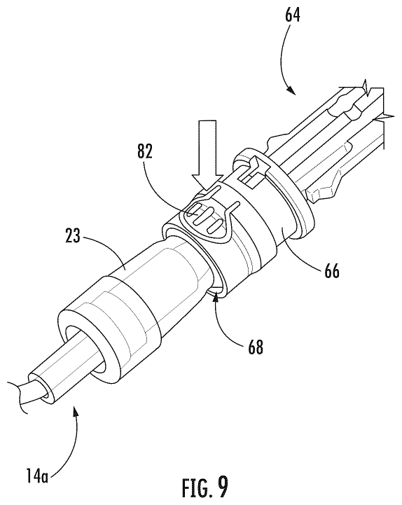

Upon initial engagement of the male luer connector 68 with the female luer connection 24, a twisting disconnection of the connector 64 from the IV line 14a is prevented. Specifically, application of a twisting force/motion to the luer housing 66 of connector 64 in the counterclockwise direction will not cause the male luer connector 68 to disconnect from the female luer connection 24 of needle-free connector 23, as shown in FIG. 8. That is, due to a slipping of the pawl teeth 84 on luer housing 66 over the ratchet teeth 104 on the male luer connector 68 (FIG. 6) that occurs responsive to the counterclockwise twisting force/motion, the male luer connector 68 rotates freely relative to the luer housing 66, which prevents disconnection of the male luer connector 68 from the female luer connection 24, as any rotation luer housing 66 of connector 64 is not transferred to male luer connector 68—resulting in the male luer connector 68 remaining engaged/secured to female luer connection 24.

As shown in FIG. 9, when it is desired to separate the connector 64 from the IV line 14a, a patient or healthcare provider applies a pinching force to the finger tabs 82 of connector 64 in order to cause the finger tabs 82 to deflect radially inward. With the finger tabs 82 deflected radially inward, the finger tabs 82 are brought into contact with the male luer connector 68 (i.e., the stem 92 of male luer connector 68, see FIG. 4).

With the finger tabs 82 deflected radially inward and in contact with the male luer connector 68, a patient or healthcare provider may then apply a twisting force/motion to the luer housing 66 of connector 64 in the counterclockwise direction that causes the male luer connector 68 to disengage from the female luer connection 24 of needle-free connector 23—such that connector 64 may be removed from IV line 14a. According to embodiments, the twisting force/motion applied by the patient/healthcare provider may be transferred from the finger tabs 82 of luer housing 66 to the male luer connector 68 via a friction engagement between the finger tabs 82 and the collar 90 of male luer connector 68 or via engagement of the finger tabs 82 with the ribs 102 on collar 90 of the male luer connector 68.

Accordingly, with the luer housing 66 and male luer connector 68 of connector 64 structured as described above, accidental disengagement of the connector 64 from an IV line 14a (i.e., from a needle-free connector 23 attached to the IV line 14a) is prevented, while allowing for selective disengagement of the connector 64 from the from an IV line 14a when desired.

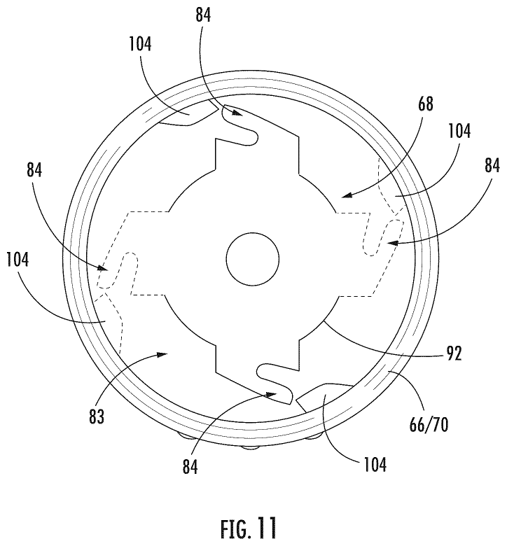

It is recognized that additional embodiments of the connector 64 may be provided that vary certain features of the connector 64, while remaining within the scope of the disclosure. Referring to FIG. 11, for example, the connector 64 is shown with the configuration of the ratchet and pawl system 83 being reversed. That is, the luer housing 66 is configured to include a plurality of ratchet teeth 104 formed thereon, while the stem 92 of male luer connector 68 includes a plurality of pawl teeth 84 thereon. As previously described, each of the ratchet teeth 104 and pawl teeth 84 may comprise angled teeth configured to interact/engage with each other to selectively provide for rotation between the male luer connector 68 and the luer housing 66 of connector 64. Specifically, the ratchet teeth 104 on luer housing 66 and the pawl teeth 84 on the male luer connector 68 each comprise angled teeth that interact to allow for rotation of the male luer connector 68 relative to the luer housing 66 in one direction but prevent rotation of the male luer connector 68 relative to the luer housing 66 in the other direction.

Beneficially, aspects of the disclosure described herein provide a connector for a CSTD that is configured to prevent accidental disconnection between a male luer connection of the connector and a female luer connection of a fluid container, adapter, or conduit attached thereto. The connector also enables a controlled disconnect of the male luer connection from the female luer connection, to allow for removal of the connector from the fluid container, adapter, or conduit.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A closed system drug transfer device connector for transferring a drug, the connector comprising:
a central shaft defining a connector lumen, the central shaft including flanges extending out radially therefrom and a pair of flexible fingers each comprising a proximal end attached to the central shaft and a free distal end configured to deflect radially inward and outward;
a seal positioned in a proximal opening of the central shaft;
a luer housing formed integrally with the central shaft and positioned at a distal end of the central shaft, the luer housing comprising a sidewall defining a cavity therein, with the sidewall including a pair of flexible finger tabs formed thereon configured to deflect radially inward responsive to a pinching force applied thereto; and
a male luer connector positioned within the cavity so as to be selectively rotatable relative to the luer housing;
wherein the male luer connector and the luer housing comprise a ratchet and pawl system configured to allow for rotation of the male luer connector relative to the luer housing when the luer housing is turned in a first direction but prevent rotation of the male luer connector relative to the luer housing when the luer housing is turned in a second direction; and
wherein, with the pair of finger tabs deflected radially inward, the pair of finger tabs engage the male luer connector to prevent rotation of the male luer connector relative to the luer housing when the luer housing is turned in the first direction.

2. The connector of claim 1, wherein the male luer connector comprises:
a distal male luer connection including an elongated protrusion and a collar surrounding the elongated protrusion;
a stem extending proximally away from the distal male luer connection; and
a luer lumen extending through the elongated protrusion and through the stem.

3. The connector of claim 2, wherein the luer housing comprises an inner surface including a first annular protrusion that defines a receptacle within the cavity, the receptacle configured to receive a proximal end of the stem therein, with the luer lumen aligned and in fluid communication with the connector lumen when the stem is positioned in the receptacle.

4. The connector of claim 3, wherein the stem includes a groove formed in an outer surface thereof, with an O-ring positioned in the groove, the O-ring forming a seal with the first annular protrusion, to provide a leak-proof connection between the connector lumen and the luer lumen.

5. The connector of claim 2, wherein the luer housing comprises an inner surface including a second annular protrusion that defines an inner lip, and wherein the male luer connector comprises an annular lip formed on the collar, with the inner lip and the annular lip providing a snap-fit connection that retains the male luer connector within the cavity, while allowing for rotation of the male luer connector relative to the luer housing.

6. The connector of claim 2, wherein the collar includes a plurality of ribs formed on an outer surface thereof, the plurality of ribs spaced circumferentially around the collar and extending radially out therefrom, and wherein the pair of finger tabs engage with a portion of the plurality of ribs when deflected radially inward.

7. The connector of claim 2, wherein the ratchet and pawl system comprises:
a plurality of pawl teeth formed on an inner surface of the luer housing at a location proximal to the pair of finger tabs; and
a plurality of ratchet teeth formed on an outer surface of the stem;
wherein the plurality of pawl teeth and the plurality of ratchet teeth each comprise angled teeth that interact to allow for rotation of the male luer connector relative to the luer housing when the luer housing is turned in the first direction but prevent rotation of the male luer connector relative to the luer housing when the luer housing is turned in the second direction.

8. The connector of claim 2, wherein the ratchet and pawl system comprises:
a plurality of ratchet teeth formed on an inner surface of the luer housing at a location proximal to the pair of finger tabs; and
a plurality of pawl teeth formed on an outer surface of the stem;
wherein the plurality of pawl teeth and the plurality of ratchet teeth each comprise angled teeth that interact to allow for rotation of the male luer connector relative to the luer housing when the luer housing is turned in the first direction but prevent rotation of the male luer connector relative to the luer housing when the luer housing is turned in the second direction.

9. The device of claim 7, wherein the plurality of pawl teeth and the plurality of ratchet teeth each comprise a pair of pawl teeth and a pair of ratchet teeth, respectively.

10. The device of claim 1, wherein each of the pair of finger tabs comprises a cantilevered tab having a first end attached to the sidewall and a free second end.

11. The device of claim 1, wherein the first direction comprises a counterclockwise direction and the second direction comprises a clockwise direction.

12. A closed system drug transfer device comprising:
the connector of claim 1; and
a fluid source, container, or conduit including a female luer connection thereon, with the female luer connection configured to engage the male luer connector to mechanically and fluidly connect the connector to the fluid source, container, or conduit.

13. The device of claim 12, wherein the prevention of rotation of the male luer connector relative to the luer housing when the luer housing is turned in the second direction enables a twisting engagement between the male luer connector and the female luer connection when the luer housing is turned in the second direction.

14. The device of claim 12, wherein with the female luer connection engaged with the male luer connector, the allowing of rotation of the male luer connector relative to the luer housing when the luer housing is turned in the first direction prevents disconnection of the female luer connection from the male luer connector.

15. The device of claim 12, wherein with the female luer connection engaged with the male luer connector and with the pair of finger tabs deflected radially inward, the pair of finger tabs engage the male luer connector to prevent rotation of the male luer connector relative to the luer housing when the luer housing is turned in the first direction, such that turning of the luer housing in the first direction enables disconnection of the female luer connection from the male luer connector.

16. The device of claim 12, wherein the fluid source, container, or conduit comprises an IV line and a needle-free connector attached thereto, with the female luer connection provided on the needle-free connector.

17. A method for assembling and disassembling the closed system drug transfer device of claim 12, the method comprising:
pushing the male luer connector of the connector into engagement with the female luer connection of the fluid source, container, or conduit; and
turning the luer housing in the second direction to thread the male luer connector into twisting engagement with the female luer connection.

18. The method of claim 17, wherein with the female luer connection engaged with the male luer connector, the method further comprises turning the luer housing in the first direction, with the male luer connector rotating freely relative to the luer housing when the luer housing is turned in the first direction, such that disconnection of the female luer connection from the male luer connector is prevented.

19. The method of claim 17, wherein with the female luer connection engaged with the male luer connector, the method further comprises:
applying a pinching force to the pair of finger tabs to cause the pair of finger tabs to deflect radially inward; and
with the pair of finger tabs deflected radially inward, turning the luer housing in the first direction, with the pair of finger tabs engaging the male luer connector to prevent rotation of the male luer connector relative to the luer housing, such that turning of the luer housing in the first direction disconnects the female luer connection from the male luer connector.
